(19) United States
(12) Patent Application Publication
(10) Pub. No.: US 2015/0005833 A1
(43) Pub. Date: Jan. 1, 2015

(54) BONE CUTTING SCREW

(71) Applicant: Howmedica Osteonics Corp., Mahwah, NJ (US)

(72) Inventor: Thomas Francis McCarthy, Neshanic Station, NJ (US)

(21) Appl. No.: 14/305,171

(22) Filed: Jun. 16, 2014

Related U.S. Application Data
(60) Provisional application No. 61/839,944, filed on Jun. 27, 2013.

(51) Int. Cl. A61B 17/86

(52) U.S. Cl. CPC A61B 17/8605
USPC 606/308

(57) ABSTRACT

A bone fastener for use in orthopedic surgery for fixing an implant to bone includes a shaft and a head. The shaft is configured to drive into bone when rotated in a first direction. The head is formed on one end of the shaft and has proximal and distal sections. The head also has at least one cutting flute on the distal section of the head. The bone fastener may be used with a prosthetic implant, such as an acetabular shell or bone plate. In use, the bone fastener is driven into the bone through a hole in the prosthetic implant, with at least a portion of the head protruding beyond the hole of the implant. As the head is driven beyond the hole of the implant, the cutting flutes in the head help secure the head of the bone fastener into the bone, increasing the stability of the system.

### CROSS-REFERENCE TO RELATED APPLICATIONS

The present application claims the benefit of the filing date of U.S. Provisional Patent Application No. 61/839,944 filed Jun. 27, 2013, the disclosure of which is hereby incorporated by reference herein.

### BACKGROUND

The present disclosure relates generally to orthopedics and, in particular, to bone screws for prosthetic implants.

In prosthetic implants, such as acetabular shells used for hip replacement or bone plates for fixing bones, bone screws are often used to fix the implant to the bone(s). Existing bone screws have screw heads that are generally contained within the wall thickness of the implant, such as within the acetabular shell or within the bone plate. Thus, the thickness of the implant is, in part, dictated by the size of the screw head, which may be dictated by the size of the connection to a driver with suitable strength for anticipated torque. For example, a large screw head may require a relatively thick bone plate or acetabular shell.

There are times when it is beneficial to utilize a smaller and/or thinner implant, such as a relatively thin acetabular shell in a revision surgery or a relatively low-profile bone plate in areas having smaller bones (e.g. on the face or cranium). In such a case, a relatively large screw head may still be desired based, for example, on the anticipated torque to be applied to the screw head. However, the use of both a relatively large screw head and a relatively thin or low-profile prosthesis may cause problems when traditional bone screws are used. For example, if the screw head of a traditional bone screw were inserted through a screw hole in a relatively thin bone plate, the screw head would protrude beyond the bottom of the bone plate and make contact with the bone, potentially causing a loss of stability or contact between the bone and the bone plate. Alternatively, the screw head could protrude beyond the non-bone contacting surface of the implant causing irritation of soft tissue or interfering with assembly of other implant components.

### BRIEF SUMMARY

In one embodiment, a bone fastener for use in orthopedic surgery for fixing an implant to bone includes a shaft and a head. The shaft may be configured to drive into the bone when rotated in a first direction. The head may be formed on one end of the shaft and have a proximal section and a distal section and at least one cutting flute suitable for cutting bone on the distal section of the head.

The head may be part spherical. The bone fastener may be a screw. The at least one cutting flute may have a first cutting surface configured to cut when rotated in the first direction. The at least one cutting flute may also have a second cutting surface configured to cut when rotated in a second direction. The at least one cutting flute may have a cavity for storing bone removed during cutting. The head may include six cutting flutes circumferentially spaced around the head.

In another embodiment, a system for use in orthopedic surgery includes a prosthetic implant and a bone fastener. The prosthetic implant may have at least one hole extending between a first side of the implant and a bone-contacting side of the implant. The bone fastener may have a shaft configured to drive into a bone when rotated in a first direction and a head formed on one end of the shaft. The head may have a proximal section and a distal section and at least one cutting flute suitable for cutting bone on the distal section of the head.

The head, when the system is implanted into the bone, may be configured to protrude beyond the bone-contacting side of the implant. The head may be part spherical. The bone fastener may be a screw. The at least one cutting flute may have a first cutting surface configured to cut when rotated in the first direction. The at least one cutting flute may also have a second cutting surface configured to cut when rotated in a second direction. The head may include six cutting flutes circumferentially spaced around the head. The prosthetic implant may be an acetabular shell. The prosthetic implant may be a bone plate.

In another embodiment, a method of performing surgery includes providing a bone fastener having a shaft configured to drive into a bone when rotated in a first direction and a head formed on one end of the shaft, the head having a proximal section and a distal section and at least one cutting flute on the distal section of the head. The method may further include the step of driving the bone fastener into the bone through a hole in a prosthetic implant, the hole extending between a first side of the implant and a bone-contacting side of the implant, until at least a portion of the head is positioned in the bone. The prosthetic implant may be an acetabular shell or a bone plate. The bone fastener may be a screw and may have a part spherical head. The at least one cutting flute may have a first cutting surface configured to cut when rotated in the first direction. The at least one cutting flute may also have a second cutting surface configured to cut when rotated in a second direction.

### BRIEF DESCRIPTION OF THE DRAWINGS

FIG. 1A is a cross-sectional view of an acetabular shell with a bone screw according to the prior art.

FIG. 1B is an enlarged cross-sectional view of the bone screw of FIG. 1A shown in the shell and a bone.

FIG. 2A is a cross-sectional view of an alternate embodiment of an acetabular shell with a bone screw according to the prior art.

FIG. 2B is an enlarged cross-sectional view of the bone screw of FIG. 2A shown in the shell and a bone.

FIG. 3A, in accordance with an aspect of the invention, is a side view of a bone screw inserted into an acetabular shell.

FIG. 3B is a side view similar to FIG. 3A with a portion of the shell in partial phantom lines.

FIG. 3C is a cross-sectional view of the bone screw of FIGS. 3A-B within the shell.

FIG. 4A is a perspective view focusing on an upper portion of the bone screw of FIGS. 3A-C.

FIG. 4B is a top plan view of the bone screw of FIGS. 3A-C with cutting flutes shown in phantom lines.

FIG. 5 is a side view of the bone screw and acetabular shell of FIGS. 3A-C in relation to a bone, the bone and acetabular shell being shown in cross section.

FIG. 6 is a side view of the bone screw of FIGS. 4A-B implanted into bone through a bone plate, the bone and bone plate being shown in cross section.

FIG. 7 is a perspective view of an upper portion of a bone screw according to another aspect of the invention.

### DETAILED DESCRIPTION

When used herein, the terms “proximal” and “distal” are used with respect to the frame of reference of a user, such as a surgeon, when using the device as intended. Specifically, the term “proximal” refers to a location on a device that is relatively close to the user, while the term “distal” refers to a location on a device that is relatively far from the user.

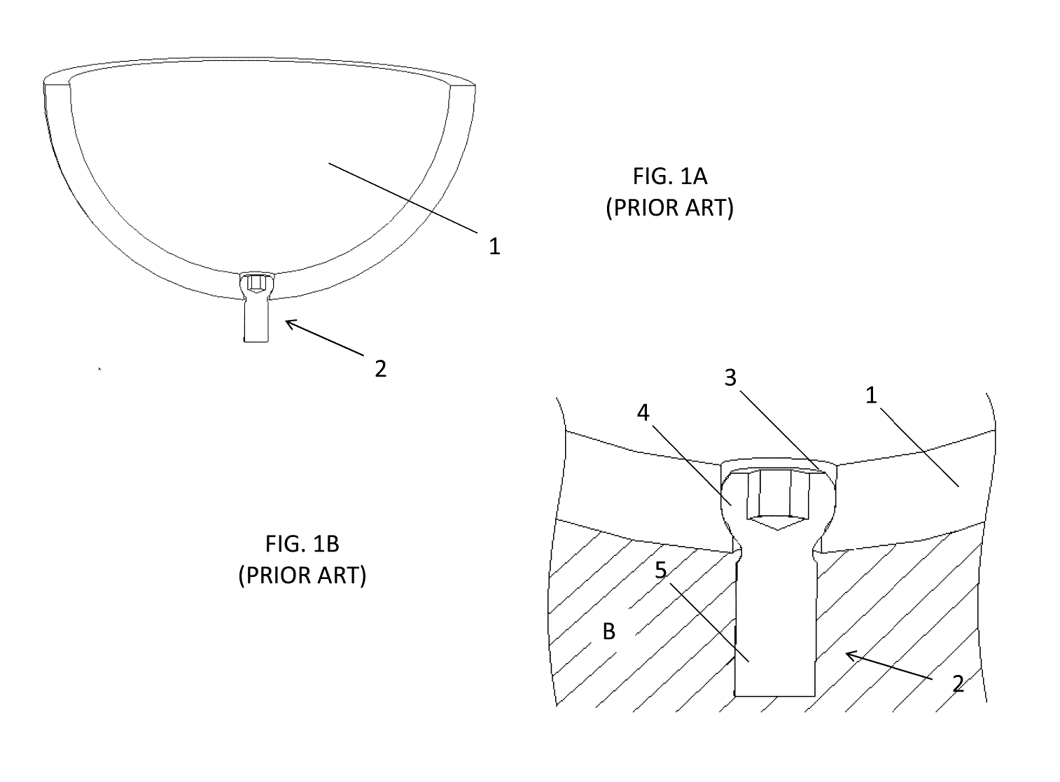

Referring to FIGS. 1A-B, an acetabular shell 1 with a bone screw 2 is shown according to the prior art. Although the disclosure illustrates a bone screw 2 with respect to an acetabular shell 1, it should be understood that the concepts illustrated herein apply equally to bone screws for other prosthetic implants, including, but not limited to, bone plates. The bone screw 2 is inserted into a bone screw hole 3 in the shell 1. The bone screw 2 includes a head 4 and a shaft 5. In this particular embodiment, the screw head 4 is part spherical. In the illustrated configuration, the shaft 5 extends through the bone screw hole 3 and beyond the shell 1. In an implanted shell 1, the shaft 5 would be implanted into bone B as illustrated in FIG. 1B. The head 4 is contained within the hole 3 in the shell 1, with little or no portion of the head 4 protruding beyond the hole 3 in the shell 1.

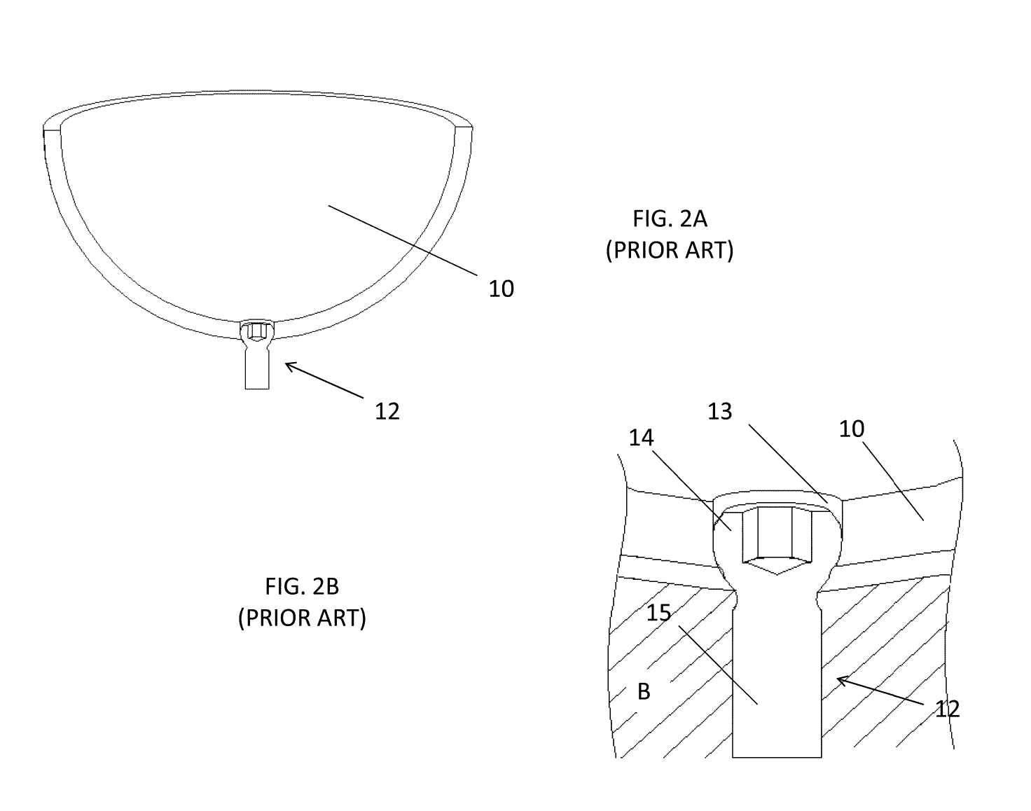

As discussed above, a thinner or lower profile implant or prosthesis may require a portion of the head of a bone screw to protrude beyond the bone-contacting surface of a prosthesis or implant. With reference to FIGS. 2A-B, an alternate embodiment of an acetabular shell 10 and bone screw is shown according to the prior art. This embodiment illustrates a traditional bone screw 12 with a shaft 15 passing through a bone screw hole 13 of a thinner shell 10. In this case, the head 14, which is shown being part spherical but may exhibit other shapes, significantly protrudes beyond the bone-contacting surface of the shell 10. As the bone screw 12 is driven into the bone B, the shaft 15 enters the bone but the portion of the screw head 14 protruding beyond the shell 10 may not, as illustrated in FIG. 2B. In this configuration, the additional space between the bone and the shell 10 created by the portion of the screw head 14 protruding beyond the shell may decrease the stability of the implant.

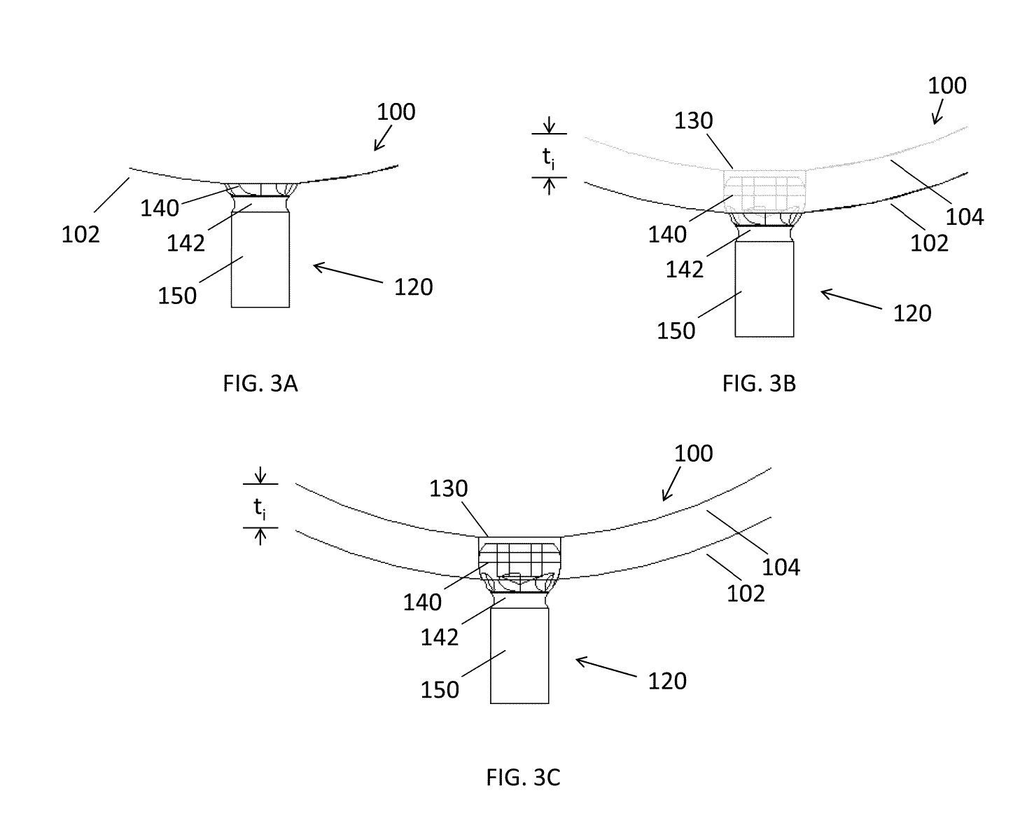

FIGS. 3A-C show a bone fastener, in the form of a bone screw 120, and a prosthetic implant, in the form of an acetabular shell 100, according to an aspect of the invention. It should be noted that FIGS. 3A-C each illustrate the same components, but with different levels of transparency. The shell 100 includes a bone-contacting surface 102 and a remote surface 104, defining an implant thickness tᵢ. The shell 100 includes at least one hole 130. It should be noted that, although an acetabular shell 100 is illustrated, the bone screw 120 or a similar screw according to another aspect of the present invention may be used with a variety of other prosthetic implants, such as bone plates.

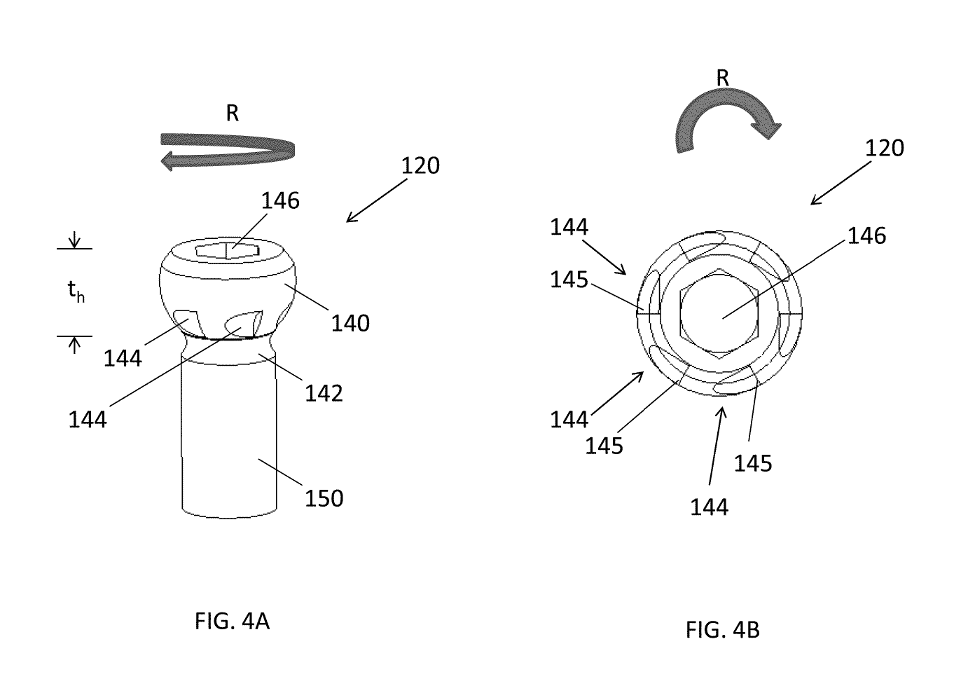

Now referring to FIGS. 3A-C and 4A-B, the bone screw 120 may include a head 140 connected to a shaft 150 by a neck portion 142. The head 140 may be part spherical and may have a head height tₕ. The head 140 may include a proximal section and a distal section. A portion of the head 140 may include one or more cutting flutes 144 suitable for cutting bone. The cutting flutes 144 may be located on only a portion, for example only on the distal section, of the head 140. Preferably, the cutting flutes 144 are arranged circumferentially around the head 140 and are equally spaced. As best illustrated in FIGS. 4A-B, the head 140 may also include a socket 146, such as a hex socket, configured to mate with a tool, such as a screwdriver (not illustrated), for driving the bone screw 120 into bone. Of course, any suitable tool mating structure may be employed. The shaft 150 of the bone screw 120 may be equipped with threads (not shown) such that the bone screw 120 drives into the bone when rotated in the direction R, and a distal tip of the shaft may be designed to facilitate tapping into bone. The cutting flutes 144 may be configured such that each cutting flute 144 has a cutting surface 145 which cuts when rotated in the direction R, which is the same direction of rotation for advancing the bone screw 120. Each flute 144 may also include a cavity (not labeled) allowing for bone accumulation during the cutting action caused by the cutting surfaces 145 of the flutes 144 during insertion of the bone screw 120 into bone.

In the illustrated embodiment, the head 140 includes six cutting flutes 140 spaced 60° apart. Other cutting flute configurations may also be utilized. Preferably, the cutting flutes 140 are configured such that they leave a portion of the surface of the screw head 140 available to interact with the internal surface of the bone screw hole 130 in the shell 100 in much the same way that a traditional screw without flutes would. In the example shown, the space between the flutes 144 maintains a spherical seat that can likewise seat into a thicker shell (not shown) in the case in which cutting flutes 144 are not desired or not necessary.

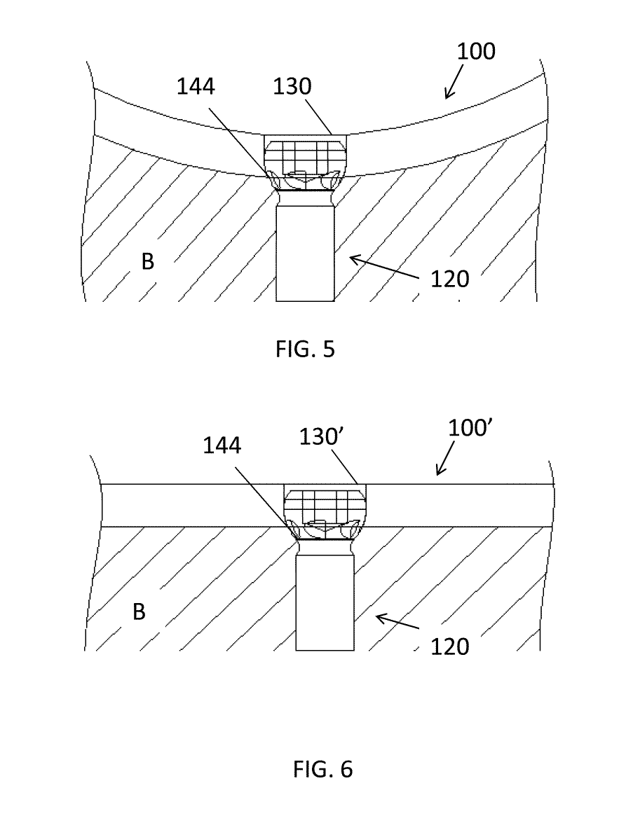

FIG. 5 illustrates the bone screw 120 implanted into bone B through the hole 130 of the acetabular shell 100. As described above, the cutting flutes 144 allow the portion of the head 140 with cutting flutes 144 to cut into the bone B. As can be seen by comparing FIG. 5 with FIG. 2B, the cutting flutes 144 allow a portion of the head 140 to ultimately be disposed within the bone B, allowing for a better fit and better stability between the acetabular shell 100 and the bone B. FIG. 6 illustrates the bone screw 120 implanted into a different prosthetic implant in the form of bone plate 100′. Similar to the embodiment with an acetabular shell 100, the bone screw 120 is implanted into bone B through a hole 130′ of the bone plate 100′. The cutting flutes 144 allow the portion of the head 140 with cutting flutes 144 to be within the bone B and the plate 100′ to be better seated on the bone. It should be noted that traditional bone screws may include machine threading on a distal portion of the head, but such threading is not suitable to cut into bone and would not be a suitable replacement for the cutting flutes of the present invention.

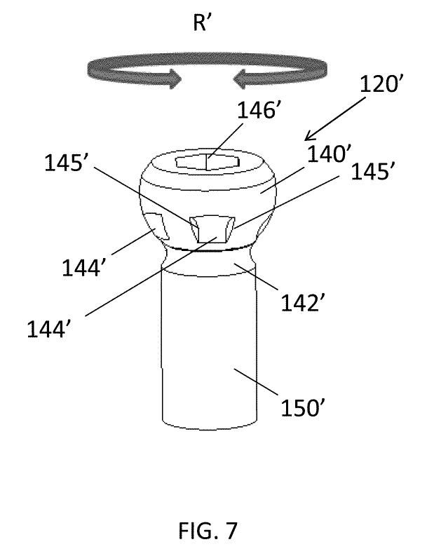

A bone screw 120′ according to an additional aspect of the invention is illustrated in FIG. 7. Similar to other embodiments described herein, the bone screw 120′ may include a head 140′ connected to a shaft 150′ by a neck portion 142′. The head 140′ may be part spherical and may include a proximal section and a distal section. A portion of the head 140′ may include one or more cutting flutes 144′. The cutting flutes 144′ may be located on only a portion, for example only on the distal section, of the head 140′. Preferably, the cutting flutes 144′ are arranged circumferentially around the head 140′ and are equally spaced. The head 140′ may also include a socket 146′, such as a hex socket, configured to mate with a tool, such as a screwdriver (not illustrated), for driving the bone screw 120′ into or out of bone. Of course, any suitable tool mating structure may be employed. The shaft 150′ of the bone screw 120′ may be equipped with threads (not shown) such that the bone screw 120′ drives into the bone when rotated in a first direction, and a distal tip of the shaft may be designed to facilitate tapping into bone.

The cutting flutes 144′ may be configured such that one or more of the cutting flutes 144′ have two cutting surface 145′ which cut when rotated in either direction R′. Thus, the cutting flutes 144′ will facilitate insertion of the bone screw 120′ into the bone as in other embodiments when rotated in the first direction to drive the bone screw 120′ into bone. If the bone screw 120′ is to be removed, for example after remaining in the bone for a period of time, the removal may be difficult due to, for example, growth of bone in and/or around the bone screw 120′. Rotation in a second direction to remove the bone screw 120′ from the bone will facilitate removal of the bone screw 120′ from the bone, for example by providing for a cutting action when rotated in the second direction. As in other embodiments described herein, each flute 144′ may also include a cavity (not labeled) allowing for bone accumulation during the cutting action caused by the cutting surfaces 145′ of the flutes 144′ during insertion and/or removal of the bone screw 120′ with respect to the bone.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. It will be appreciated that the various dependent claims and the features set forth therein can be combined in different ways than presented in the initial claims. It will also be appreciated that the features described in connection with individual embodiments may be shared with others of the described embodiments.

1. A bone fastener for use in orthopedic surgery for fixing an implant to bone, comprising:
a shaft configured to drive into the bone when rotated in a first direction; and
a head formed on one end of the shaft, the head having a proximal section and a distal section and at least one cutting flute on the distal section of the head.

2. The bone fastener of claim 1, wherein the head is part spherical.

3. The bone fastener of claim 1, wherein the bone fastener is a screw.

4. The bone fastener of claim 1, wherein the at least one cutting flute has a first cutting surface configured to cut when rotated in the first direction.

5. The bone fastener of claim 4, wherein the at least one cutting flute has a second cutting surface configured to cut when rotated in a second direction opposite the first direction.

6. The bone fastener of claim 4, wherein the at least one cutting flute has a cavity for storing bone removed during cutting.

7. The bone fastener of claim 6, wherein the head includes six cutting flutes circumferentially spaced around the head.

8. A system for use in orthopedic surgery comprising:
a prosthetic implant having at least one hole extending between a first side of the implant and a bone-contacting side of the implant; and
a bone fastener having a shaft configured to drive into a bone when rotated in a first direction and a head formed on one end of the shaft, the head having a proximal section and a distal section and at least one cutting flute on the distal section of the head
wherein the head, when the system is implanted into the bone, is configured to protrude beyond the bone-contacting side of the implant.

9. The system of claim 8, wherein the head is part spherical.

10. The system of claim 8, wherein the bone fastener is a screw.

11. The system of claim 8, wherein the at least one cutting flute has a first cutting surface configured to cut when rotated in the first direction.

12. The system of claim 11, wherein the at least one cutting flute has a second cutting surface configured to cut when rotated in a second direction opposite the first direction.

13. The system of claim 8, wherein the head includes six cutting flutes circumferentially spaced around the head.

14. The system of claim 8, wherein the prosthetic implant is an acetabular shell or a bone plate.

15. A method of performing surgery comprising:
providing a bone fastener having a shaft configured to drive into a bone when rotated in a first direction and a head formed on one end of the shaft, the head having a proximal section and a distal section and at least one cutting flute on the distal section of the head; and
driving the bone fastener into the bone through a hole in a prosthetic implant, the hole extending between a first side of the implant and a bone-contacting side of the implant, until at least a portion of the head is positioned in the bone.

16. The method of claim 15, wherein the prosthetic implant is an acetabular shell or a bone plate.

17. The method of claim 15, wherein the bone fastener is a screw.

18. The method of claim 17, wherein the head is part spherical.

19. The method of claim 15, wherein the at least one cutting flute has a first cutting surface configured to cut when rotated in the first direction.

20. The method of claim 19, wherein the at least one cutting flute has a second cutting surface configured to cut when rotated in a second direction opposite the first direction.
